(19) United States
(12) Patent Application Publication
(10) Pub. No.: US 2019/0142295 A1
(43) Pub. Date: May 16, 2019

(54) ELECTRODE, BIOELECTRODE, AND MANUFACTURING METHOD THEREOF

(71) Applicant: TATSUTA ELECTRIC WIRE & CABLE CO., LTD., Osaka (JP)

(72) Inventors: Keisho SHINOHARA, Kyoto (JP); Satoru ONIZUKA, Kyoto (JP)

(21) Appl. No.: 16/175,065

(22) Filed: Oct. 30, 2018

(30) Foreign Application Priority Data
Nov. 16, 2017 (JP) 2017-220762
Jun. 1, 2018 (JP) 2018-106348

(51) Int. Cl. A61B 5/0408; A61B 5/0478; A61N 1/04

(52) U.S. Cl. CPC A61B 5/04087; A61B 5/0478; A61N 1/0472; A61B 2562/125; A61B 2562/0215; A61B 2562/0217; A61N 1/042

(57) ABSTRACT

Provided is an electrode comprising a sheet material, wherein the sheet material comprises a conductive portion which is continuous so that the sheet material has electrical conductivity in a thickness direction thereof, wherein the conductive portion comprises an electrical conductor distributed in the conductive portion so that the sheet material has electrical conductivity in the thickness direction thereof.

### CROSS-REFERENCE TO RELATED APPLICATION

This application claims priority from Japanese Patent Application No. 2017-220762 filed with the Japan Patent Office on Nov. 16, 2017, and Japanese Patent Application No. 2018-106348 filed with the Japan Patent Office on Jun. 1, 2018, the entire contents of which are hereby incorporated by reference.

### BACKGROUND

### 1. Technical Field

The present invention relates to an electrode, a bioelectrode, and a manufacturing method thereof.

### 2. Related Art

Various manufacturing methods of the bioelectrode which is necessary for recording an electrocardiogram, an electromyogram, an electroencephalogram, or the like have been studied. As the electrode, an electrode including a film containing a carbon or graphite layer and a silver or silver chloride layer laminated in the following order on a first surface of the film is widely known. As the film, a non-conductive film such as a polyester film is used (for example, see JP-A-05-095922).

### SUMMARY

An electrode according to an embodiment of the present disclosure comprises a sheet material, wherein the sheet material comprises a conductive portion which is continuous so that the sheet material has electrical conductivity in a thickness direction thereof, wherein the conductive portion comprises an electrical conductor distributed in the conductive portion so that the sheet material has electrical conductivity in the thickness direction thereof.

### BRIEF DESCRIPTION OF DRAWINGS

FIG. 1 is a cross-sectional view schematically showing a structure of an electrode according to Embodiment 1 of the present disclosure;

FIG. 2 is a flowchart of a method of manufacturing the electrode according to Embodiment 1;

FIG. 3 is a cross-sectional view schematically showing a structure of an example of a bioelectrode according to Embodiment 2 of the present disclosure;

FIG. 4 is a flowchart of a method of manufacturing the bioelectrode according to Embodiment 2;

FIG. 5 is a cross-sectional view schematically showing a structure of another example of the bioelectrode;

FIG. 6 is a cross-sectional view schematically showing a structure of the electrode according to Embodiment 3 of the present disclosure;

FIG. 7 is a flowchart of a method of manufacturing the bioelectrode using the electrode according to Embodiment 3;

FIG. 8 is a cross-sectional view schematically showing a structure of the electrode according to Embodiment 4 of the present disclosure; and

FIG. 9 is a flowchart of a method of manufacturing the bioelectrode using the electrode according to Embodiment 4.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

However, in the above-described electrode of the related art, a second surface of the film on which the carbon or graphite layer and the silver or silver chloride layer are not formed is not electrically conductive. Therefore, in the above-described electrode of the related art, in order to obtain electrical conductivity between the first surface and the second surface of the film, an additional step such as drilling of the film is required.

An object of an aspect of the present disclosure is to realize the electrode, the bioelectrode, and the manufacturing method thereof, which can easily obtain electrical conductivity between the first surface and the second surface.

An electrode according to a first aspect of the present disclosure includes a sheet material, and a conductive portion which is continuous so that the sheet material has electrical conductivity in a thickness direction thereof, wherein the conductive portion comprises an electrical conductor distributed in the conductive portion so that the sheet material has electrical conductivity in the thickness direction thereof.

A bioelectrode according to a second aspect of the present disclosure includes the electrode, a conductive gel layer disposed on the conductive portion, and a lead wire electrically connected to the conductive portion.

A method of manufacturing an electrode according to a third aspect of the present disclosure includes a step of permeating a sheet material having liquid permeability with a conductive coating material containing an electrical conductor, to form a conductive portion which is continuous so that the sheet material has electrical conductivity in a thickness direction thereof.

A method of manufacturing a bioelectrode according to a fourth aspect of the present disclosure includes a step of forming a conductive gel layer on the conductive portion of the electrode manufactured by the method of manufacturing the electrode, and a step of electrically connecting a lead wire to the conductive portion.

According to the present disclosure, it is possible to conduct not only an electrode layer on the first surface of the sheet material but also the conductive portion exposed on the second surface of the sheet material. Therefore, it is possible to realize the electrode which easily conducts from both sides and the bioelectrode having such an electrode.

### Embodiment 1

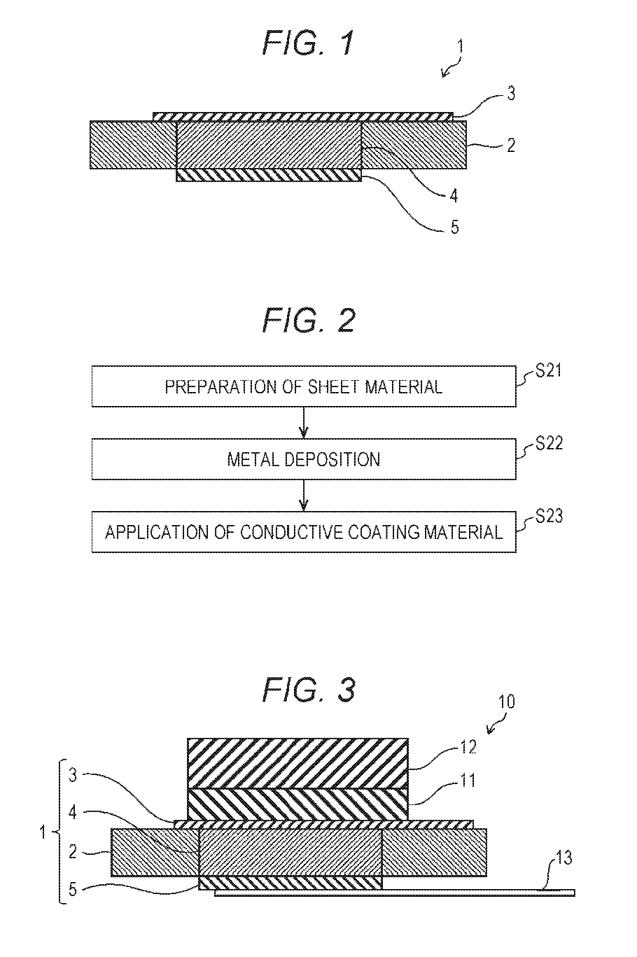

FIG. 1 is a cross-sectional view schematically showing a structure of the electrode according to Embodiment 1 of the present disclosure. As shown in FIG. 1, an electrode 1 according to Embodiment 1 has a sheet material 2 and a metal layer 3.

From a viewpoint of supporting the structure of the electrode 1 and a viewpoint of facilitating handling of the electrode 1, the sheet material 2 preferably has a thickness of 0.01 to 1.5 mm. Further, the sheet material 2 preferably has liquid permeability. The liquid permeability of the sheet material 2 may be, for example, liquid permeability such that the conductive coating material described below sufficiently penetrates. The sheet material 2 usually does not have electrical conductivity. However, the sheet material 2 may have electrical conductivity.

Examples of the sheet material 2 include a nonwoven fabric, a woven fabric, a knitted material, a paper, a synthetic resin net, and a metal net. The paper may be Japanese paper or paper.

The sheet material 2 may be the nonwoven fabric. A basis weight of the nonwoven fabric is preferably 300 g/m² or less, more preferably 200 g/m² or less from a viewpoint of sufficient penetration of the coating material when the conductive coating material described below is applied. As such a nonwoven fabric, the nonwoven fabric obtained by a known manufacturing method such as a dry method, a wet method, a spunbond method, a melt blown method, an air laid method, or the like can be appropriately used.

The material of the nonwoven fabric may be a resin. Examples of such resins include polyester, nylon, polypropylene, and polybutylene terephthalate. The nonwoven fabric may be a commercially available product. Examples of commercially available products include MALIX#20451FLV and #20457FLV (both manufactured by Unitika Ltd., “MARIX” is a registered trademark of the company) and ELTAS E5030, EC5045C, and E05050 (all manufactured by Asahi Kasei Corporation, “ELTAS” is a registered trademark of the company).

The metal layer 3 is disposed on the first surface of the sheet material 2. The metal layer 3 corresponds to a conductive layer. The material constituting the conductive layer can be appropriately selected as long as it has sufficient electrical conductivity as the electrode 1. For example, the conductive layer may be a film of a conductive composition having electrical conductivity. Such a conductive layer can be produced, for example, by applying conductive ink.

The metal layer 3 may cover an entire first surface of the sheet material 2. Alternatively, the metal layer 3 may be disposed on a part of the first surface. The number and shape of the metal layers 3 on the first surface of the sheet material 2 are not limited. For example, the metal layer 3 may be disposed so as to cover an entire predetermined region on the first surface of the sheet material 2. Alternatively, the metal layer 3 may be disposed dispersedly in the region. That is, the metal layer 3 may be disposed on the first surface of the sheet material 2 by solid printing. Alternatively, the metal layer 3 may be printed on a pattern-like portion such as a dot or a stripe.

Various conditions such as electrical conductivity, durability such as corrosion resistance, cost, and the like are taken into consideration when selecting the metal material of the metal layer 3. An appropriate metal material can be selected from a range of materials giving an effect of the present embodiment. The metal material may be one or more kinds. Examples thereof include gold, silver, copper, nickel, tin, aluminum, zinc, indium tin oxide, titanium, and the like. Among them, silver is preferable from a viewpoint of high durability, appropriate affinity for other members of the bioelectrode, and excellent X-ray permeability.

A thickness of the metal layer 3 can be appropriately determined based on, for example, a type of the metal material and a method of producing the metal layer. When the metal layer 3 is too thin, electrical contact described below between the sheet material 2 and the conductive portion in the sheet material 2 may be insufficient. When the metal layer 3 is too thick, flexibility of the electrode 1 is lowered. Therefore, handling property of the electrode 1 may be lowered in some cases. From these viewpoints, the thickness of the metal layer 3 can be selected from, for example, a range of 0.03 to 20 μm. Preferably, the thickness of the metal layer 3 is 0.1 to 2 μm. More specifically, when the metal layer 3 is a silver-deposited film, the thickness of the metal layer 3 is preferably 0.03 μm or more, more preferably 0.06 μm or more. Further, the thickness of the metal layer 3 is preferably 2 μm or less, more preferably 0.4 μm or less.

Examples of the method of producing the metal layer 3 include a plating method and a thermal transfer method in addition to vacuum deposition. From a viewpoint that the metal layer having excellent flexibility can be obtained and a viewpoint that easy mass production and merit of cost can be expected, a preferred method of producing the metal layer 3 is vacuum deposition.

The sheet material 2 includes a conductive portion 4. The conductive portion 4 is a portion having electrical conductivity. By the conductive portion 4, the sheet material 2 has electrical conductivity in the thickness direction thereof. Further, the conductive portion 4 is disposed at a position overlapping one or more specific metal layers 3 of the sheet material 2. The number and shape of the conductive portions 4 in the sheet material 2 are not limited.

For example, the conductive portion 4 may be disposed on the surface of the sheet material 2 so as to cover the entire predetermined region on the surface of the sheet material 2 similarly to the metal layer 3. Alternatively, the conductive portion 4 may be disposed dispersedly in the region. Further, the conductive portion 4 may be electrically continuous as a whole of the conductive portion 4 in the thickness direction of the sheet material 2. For example, the conductive portion may be continuous from the first surface of the sheet material 2 to the second surface of the sheet material 2 in the thickness direction of the sheet material 2.

The phrase “the conductive portion is electrically continuous” means that the conductive portion is present in the sheet material so as to have a density sufficient for desired electrical conductivity in the thickness direction of the sheet material to be obtained.

The conductive portion 4 includes an electrical conductor. The electrical conductor is distributed in the conductive portion 4 so that the sheet material 2 has electrical conductivity in the thickness direction thereof. The electrical conductor may be electrically continuously distributed in the thickness direction of the sheet material 2 regardless of distribution in a plane direction of the sheet material 2. The electrical conductor may be a substance having electrical conductivity. The electrical conductor may be particles. The phrase “the electrical conductor is electrically continuously distributed” means that the electrical conductor is present in the conductive portion so as to have a density sufficient for desired electrical conductivity in the thickness direction of the sheet material to be obtained. It is possible to appropriately realize appropriate distribution of the electrical conductor in the conductive portion 4 based on, for example, a state of the electrical conductor and liquid permeability of the sheet material 2.

The electrical conductor may be one or more kinds. Examples of the electrical conductor include an ion conductive agent and an electron conductive agent. Examples of the ion conductive agent include silver iodide, copper iodide, silver chloride, lithium perchlorate, lithium trifluoromethanesulfonate, lithium salt of organic boron complex, lithium bisimide ((CF₃SO₂)₂NLi), and lithium trismethide ((CF₃SO₂)₃CLi). Examples of the electron conductive agent include metal particles and carbon compound particles. Examples of metal of the metal particles include silver, copper, aluminum, magnesium, nickel, and stainless steel. Examples of the carbon compound include graphite, carbon black, carbon nanofiber, and carbon nanotube. From a viewpoint of corrosion resistance, the carbon compound is a preferable electrical conductor. From a viewpoint of availability, a carbon particle is a preferable electrical conductor.

The electrode 1 may further include elements other than the sheet material 2, the metal layer 3, and the conductive portion 4 described above, within a range in which the effect of the present embodiment can be obtained. For example, the electrode 1 may further include a conductive coating film 5.

The conductive coating film 5 is disposed on a surface portion on the opposite side to the metal layer 3 side of the sheet material 2 in the conductive portion 4. The conductive coating film 5 is a layer formed of the conductive composition. For example, the conductive coating film 5 is a portion remaining after the conductive coating material containing the electrical conductor penetrates into the sheet material 2. The conductive coating film 5 is formed, for example, by application of the conductive coating material in a manufacturing method described below. The conductive coating film 5 is preferable from a viewpoint of clearly indicating a position of the conductive portion 4 from the opposite side to the metal layer 3 in the sheet material 2 and from a viewpoint of improving electrical connection with the conductive portion 4.

Another layer may be interposed between the sheet material 2 and the metal layer 3 within the range in which the effect of the present embodiment can be obtained. Or, the electrode 1 may further include a layer on the metal layer 3. Any layer which can be arbitrarily added in this manner may have sufficient electrical conductivity.

The electrode 1 can be manufactured by the following method. FIG. 2 is a flowchart of a method of manufacturing the electrode 1.

First, the sheet material 2 is prepared (S21). As the sheet material 2, a commercially available product may be used as it is. Alternatively, a commercially available product cut into an appropriate shape may be used.

Next, the metal layer 3 is formed on the first surface of the sheet material 2. Examples of methods of producing the deposited film include vacuum deposition, sputtering, and ion plating. Other examples of the method of producing the metal layer 3 include a metal plating method. For example, a metal-deposited film is formed on the first surface of the sheet material 2. The metal layer 3 can be formed in an arbitrary shape at an arbitrary position on the first surface of the sheet material 2, for example, by using a known masking technique.

Next, the conductive coating material is applied to the second surface of the sheet material 2 (S23). The conductive coating material is applied to a position overlapping the metal layer 3 on the first surface of the sheet material 2. The conductive coating material can be applied to the sheet material 2 by a known coating method such as coat printing. Examples of the coating method include a gravure coating method, a die coating method, a comma coating method, a roll coating method, a dipping method, a screen method, and a rotary screen method.

The applied conductive coating material penetrates into the sheet material 2 having liquid permeability. Then, the conductive coating material crosses the sheet material 2 in the thickness direction and reaches the metal layer 3. Thus, the conductive portion 4 is formed.

The conductive coating material contains the electrical conductor. The conductive coating material may contain only the electrical conductor. Alternatively, the conductive coating material may contain the electrical conductor and a dispersion medium such as a binder. The dispersion medium may be a medium having sufficient fluidity so as to function as the dispersion medium of the conductor in the conductive coating material. The dispersion medium may be one or more kinds.

Content of the electrical conductor in the conductive coating material can be appropriately determined from a viewpoint that the sufficient electrical conductivity of the conductive portion 4 is obtained. The electrical conductivity of the conductive portion 4 can be adjusted by a coating amount, the number of coating times, or viscosity of the conductive coating material, in addition to the content of the electrical conductor in the conductive coating material.

The conductive coating material is preferably ink containing carbon particles. The conductive coating material can be obtained by appropriately preparing it. However, a commercially available conductive coating material can also be used. Examples of commercially available inks containing the carbon particles include “UCC-2” manufactured by Nippon Graphite Industries, Co., Ltd., “JELCON CH-8” manufactured by Jujo Chemical Co., Ltd., and “DY-200L-2” manufactured by Toyobo Co., Ltd.

The above manufacturing method may further include other steps than the above-mentioned steps within the range in which the effect of the present embodiment can be obtained.

When manufacturing the electrode 1, the sheet material 2 having liquid permeability is impregnated with the conductive coating material by coat printing. Thus, the conductive portion 4 extending from the second surface of the sheet material 2 to the metal layer 3 on the first surface is formed. In this manner, in the electrode 1, the electrical conductivity between the surfaces of the sheet material 2 is established by vapor deposition and coating on the surfaces of the sheet material 2 having liquid permeability. In an electrode having a film instead of the sheet material, in order to obtain the electrical conductivity between the surfaces of the film, a devisal such as drilling a hole in the film is required.

Since the electrode 1 has the above structure, it is possible to connect a terminal to the second surface of the sheet material 2. Therefore, for example, the first surface of the electrode 1 on the metal layer 3 side can be coated with various functional coating agents. As described above, in the electrode 1, degrees of freedom in structure and function of the product including the electrode 1 is high. In these respects, the electrode 1 is advantageous.

The electrode 1 is suitably used as a bioelectrode described below.

### Embodiment 2

FIG. 3 is a cross-sectional view schematically showing a structure of an example of a bioelectrode according to Embodiment 2 of the present disclosure. In this example, a bioelectrode 10 includes the electrode 1, a silver chloride layer 11 disposed on the metal layer 3, a conductive gel layer 12 disposed on the silver chloride layer 11, and a lead wire 13 electrically connected to the conductive coating film 5. The silver chloride layer 11 and the conductive gel layer 12 can be configured similarly to those of known bioelectrodes.

The silver chloride layer 11 is a layer containing silver chloride. A thickness of the silver chloride layer 11 is preferably 3 to 30 μm from a viewpoint of stability of ionic conduction. Content of silver chloride in the silver chloride layer 11 can be appropriately determined from a range in which a desired function as the bioelectrode can be obtained. The content is, for example, 1 to 10 mass %.

The conductive gel layer 12 is a layer containing conductive gel. A thickness of the conductive gel layer 12 can be appropriately determined within a range in which sufficient adhesion to a living body and necessary electrical conductivity to the living body can be obtained. The thickness is, for example, 0.5 to 2 mm. As the conductive gel, it is possible to use, for example, a known self-adhesive conductive gel having skin compatibility.

The conductive gel normally contains the gel and an electrolyte. Examples of the gels include the gel made of a composition containing a crosslinked copolymer of alkoxypolyethylene glycol mono (meth)acrylate and an unsaturated carboxylic acid such as acrylic acid, polyethylene glycol monoalkyl ether, and water. Incidentally, the unsaturated carboxylic acid may be partially neutralized.

Examples of the electrolyte include alkali metal halides such as lithium chloride, sodium chloride, and potassium chloride. The conductive gel may further contain other components within a range in which the above-described adhesiveness and electrical conductivity can be obtained. Examples of the other components include a moisturizing component, a perfume, a coloring agent, and a medicinal component. Examples of the moisturizing components include lactic acid, urea, and hyaluronic acid.

The numbers and shapes of the silver chloride layers 11 and the conductive gel layers 12 are not limited. For example, the shapes thereof in a plan view can be appropriately determined within a range in which they can be used for application of the bioelectrode. The shapes of the silver chloride layer 11 and the conductive gel layer 12 may be the same or different.

The lead wire 13 may be an ordinary lead wire having a conductive wire such as a carbon wire, a tin-plated Cu wire, an A1 wire, and an insulating layer covering the lead wire. The number of the lead wires 13 is not limited. The number of the conductive portions 4 to which the lead wires 13 are connected is neither limited.

The bioelectrode 10 can be applied as the electrode for measuring an electrocardiogram. Examples of the electrodes for measuring the electrocardiogram include a disposable pad electrode used in a defibrillator. Examples of the defibrillators include an external defibrillator, a semi-automatic defibrillator, and an automatic external defibrillator (AED).

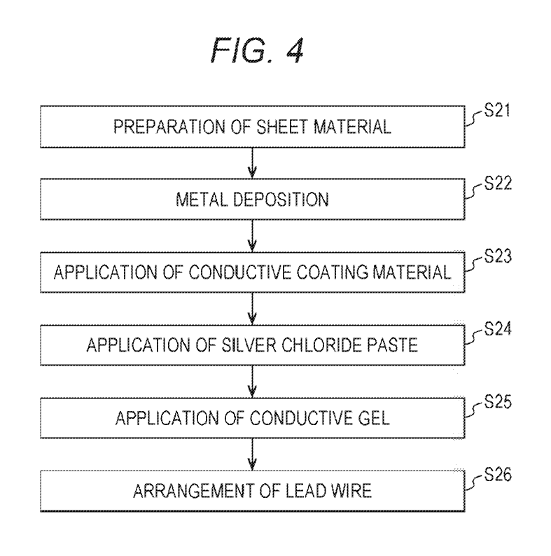

The bioelectrode 10 can be manufactured by the following method. FIG. 4 is a flowchart of a method of manufacturing the bioelectrode 10.

In manufacturing the bioelectrode 10, as shown in FIG. 4, the silver chloride layer 11 is formed on the metal layer 3 of the electrode 1 manufactured by the method of manufacturing the electrode 1 (S24). As described above, the electrode 1 can be manufactured, for example, by Step S21 to Step S23. The silver chloride layer 11 can be produced by a known method. For example, the silver chloride layer 11 can be formed by printing a coating agent containing silver chloride on the surface of the metal layer 3 uniformly to a desired thickness using the known printing technique such the a die coating method, the comma coating method, or the dipping method.

Next, the conductive gel layer 12 is formed on the formed silver chloride layer 11 (S25). The conductive gel layer 12 can also be produced by a known method. For example, the conductive gel layer 12 may be formed on the surface of the silver chloride layer 11 in the same manner as the silver chloride layer 11 by application or printing. Or, the conductive gel layer 12 may be formed by attaching a conductive gel layer formed on a releasing film to the silver chloride layer 11 and then peeling off the releasing film as necessary.

Meanwhile, the lead wire 13 is electrically connected to the conductive portion 4 (S26). However, the lead wire 13 can be electrically connected to the conductive portion 4 at an arbitrary timing after producing the conductive portion 4. The lead wire 13 can be electrically connected to the conductive portion 4 by a known method of electrically connecting the conductive wire of the lead wire 13 to the conductive portion 4. In this electrical connection, another member such as a conductive fastener fixed to the surface of the conductive portion 4 may be used. A device (for example, the defibrillator described above) connected to the bioelectrode or a connector for connecting to the device may be connected to an end portion of the lead wire 13 on the opposite side to a connection end of the conductive portion 4.

As described above, according to the present embodiment, it is possible to transmit and receive electrical signals from the entire second surface of the sheet material 2 by the conductive coating material containing the carbon particles printed on the sheet material 2. Therefore, it is possible to further increase a connection area of the lead wire 13. Therefore, in the bioelectrode 10, detection sensitivity of a bioelectrical signal can be further increased as compared with the conventional bioelectrode. Further, a response speed can be further increased. Furthermore, the bioelectrode 10 having excellent reversibility of electrode potential is provided. Thus, electrical characteristics of the bioelectrode 10 can be further improved (impedance can be reduced).

The sheet material 2 has liquid permeability. Therefore, the sheet material 2 is generally rich in flexibility. Therefore, the adhesion of the bioelectrode 10 to the living body can be further increased.

### [Modification of Embodiments 1 and 2]

In the conductive portion 4 of the electrode 1, the carbon particles are used as the electrical conductor. However, the electrical conductor may contain metal powders together with the carbon particles.

In the method of manufacturing the electrode 1, the metal layer 3 is formed prior to the conductive portion 4. However, the metal layer 3 may be formed after the conductive portion 4 so that the metal layer 3 and the conductive portion 4 are positioned to overlap each other when viewed from the thickness direction of the sheet material 2.

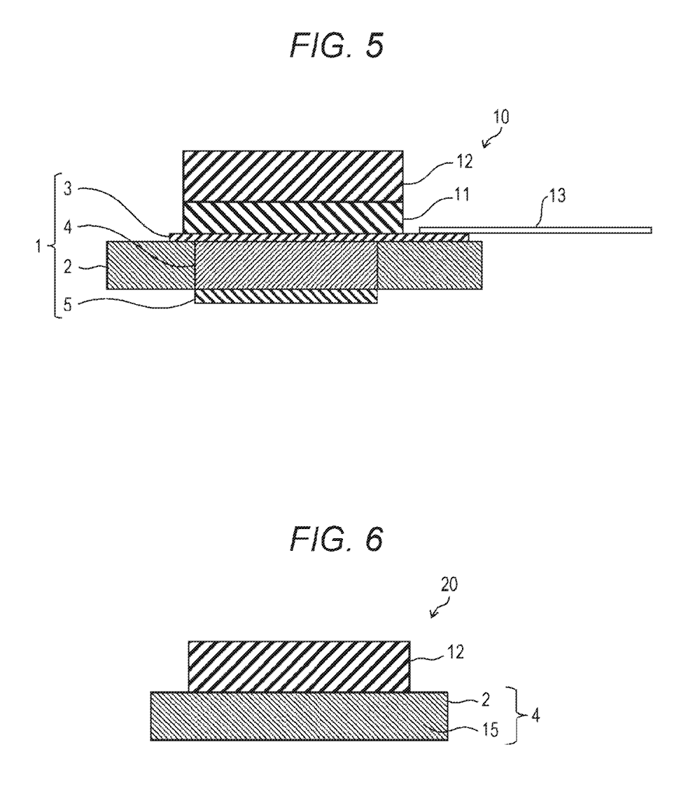

In the bioelectrode 10, as shown in FIG. 5, the lead wire 13 may be electrically connected to the metal layer 3. Further, the lead wire 13 may be electrically connected to both the conductive portion 4 and the metal layer 3.

### [Summary of Embodiments 1 and 2]

The electrode according to an aspect 1 of the present disclosure includes the sheet material and the conductive layer disposed on the sheet material. The sheet material includes the conductive portion which is continuous so that the sheet material has electrical conductivity in the thickness direction thereof. The conductive portion includes the electrical conductor distributed in the conductive portion so that the sheet material has electrical conductivity in the thickness direction thereof.

According to the above structure, the conductive portion is also exposed on the surface (the other surface) not having the conductive layer of the sheet material. Therefore, it is possible to easily conduct from both the conductive layer on one surface side of the sheet material and the conductive portion on the other surface side of the sheet material.

In the electrode according to an aspect 2 of the present disclosure, the electrical conductor of the aspect 1 may be the carbon particles.

The above structure is more effective from the viewpoint of improving the corrosion resistance of the electrode.

In the electrode according to an aspect 3 of the present disclosure, the conductive layer of Embodiment 1 or 2 may include the metal layer.

The above structure is more effective from the viewpoint of improving durability and productivity of the electrode.

In the electrode according to an aspect 4 of the present disclosure, the metal layer of the aspect 3 may be the metal-deposited layer.

The above structure is more effective from the viewpoint of realizing the metal layer having a desired shape, for example, a precise pattern.

In the electrode according to an aspect 5 of the present disclosure, the metal material of the metal layer of any one of aspects 1 to 4 may be one or more metal materials selected from the group consisting of gold, silver, copper, nickel, tin, aluminum, zinc, indium tin oxide, and titanium.

The above structure is more effective from the viewpoint of improving the corrosion resistance of the electrode or from the viewpoint of reducing the manufacturing cost of the electrode.

In the electrode according to an aspect 6 of the present disclosure, the sheet material of the aspect 1 may be one or more members selected from the group consisting of the nonwoven fabric, the woven fabric, the knitted material, the paper, the synthetic resin net, and the metal net.

The above structure is more effective from the viewpoint of improving strength of the electrode and ease of handling.

The bioelectrode according to an aspect 7 of the present disclosure includes the electrode according to any one of the above-described embodiments, the silver chloride layer disposed on the conductive layer, the conductive gel layer disposed on the silver chloride layer, and the lead wire electrically connected to the conductive layer or the conductive portion.

According to the above structure, the conductive portion is also exposed on the surface not having the conductive layer of the sheet material. Therefore, it is possible to connect the lead wire to any of a plurality of surfaces of the sheet material, that is, to both the conductive layer and the conductive portion. Therefore, the degree of freedom in the structure of the bioelectrode is increased.

The bioelectrode according to an aspect 8 of the present disclosure may be the electrode of the aspect 6 for measuring the electrocardiogram.

A method of manufacturing the electrode according to an aspect 9 of the present disclosure includes the steps of forming the conductive layer on the surface of the sheet material having liquid permeability, and forming the conductive portion so that the sheet material has electrical conductivity in the thickness direction by penetrating the conductive coating material containing the electrical conductor at a position overlapping the conductive layer in the sheet material.

According to the above structure, it is possible to manufacture the electrode which can easily conduct from both the conductive layer on the first surface of the sheet material and the conductive portion on the second surface of the sheet material.

In the method of manufacturing the electrode according to an aspect 10 of the present disclosure, the ink containing the carbon particles can be used as the conductive coating material in the manufacturing method of the aspect 8.

The above structure is more effective from the viewpoint of improving the corrosion resistance of the electrode.

A method for manufacturing the bioelectrode according to an aspect 11 of the present disclosure includes the steps of forming the silver chloride layer on the conductive layer of the electrode manufactured by the method for manufacturing the electrode of the above embodiment, forming the conductive gel layer on the formed silver chloride layer, and electrically connecting the lead wire to the conductive layer or the conductive portion.

According to the above structure, it is possible to manufacture the bioelectrode which can be connected to both the conductive layer and the conductive portion and has a high degree of freedom in the structure.

### Embodiment 3

Further embodiments of the present disclosure will be described below. Matters overlapping those of the above-described embodiments may not be described again.

FIG. 6 is a cross-sectional view schematically showing the structure of the electrode according to Embodiment 3. An electrode 20 has the sheet material 2 and the conductive gel layer 12. The sheet material 2 is impregnated with the conductive coating material 15. A portion impregnated with the conductive coating material 15 of the sheet material 2 corresponds to the conductive portion 4.

The conductive coating material 15 includes the electrical conductor having good affinity for the conductive gel layer 12. For example, when the conductive gel layer 12 contains the above-described electrolyte, the conductive coating material 15 contains the above-described silver halide as the electrical conductor. In the above case, the content of the silver halide in the conductive coating material 15 can be appropriately determined within a range in which the conductive portion 4 exhibits sufficient affinity to the conductive gel layer 12 and sufficient electrical conductivity. The electrical conductor may be one kind or may further include other electrical conductors. The type and amount of the other electrical conductors can be appropriately determined, for example, from the viewpoint of exhibiting sufficient electrical conductivity in the conductive portion 4.

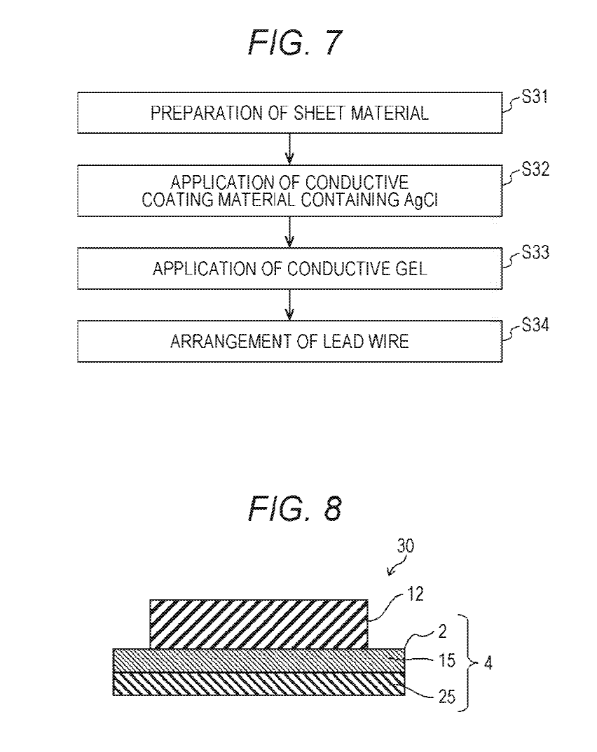

In the present embodiment, the bioelectrode can be manufactured by the following method. FIG. 7 is a flowchart of the method of manufacturing the bioelectrode using the electrode 20.

First, the sheet material 2 is prepared (S31). It is possible to prepare the sheet material 2 in the same manner as in Step S21 described above.

Next, the conductive coating material containing silver chloride (AgCl-containing conductive coating material) is applied to the sheet material 2 (S32). A method of applying the conductive coating material can be appropriately determined from a known coating method within a range in which the conductive portion can be formed so that the sheet material 2 has sufficient electrical conductivity in the thickness direction.

Next, the conductive gel is applied to the surface of the sheet material 2 impregnated with the conductive coating material to prepare the conductive gel layer 12 (S33). The conductive gel can be applied in the same manner as in Step S25 described above.

Next, the lead wire is placed on the sheet material 2 (S34). A connection position of the lead wire on the sheet material 2 can be appropriately determined at an arbitrary position in the conductive portion 4. For example, the connection position may be a portion around the conductive gel layer 12 on the first surface of the sheet material 2. Further, the connection position may be on the second surface (surface on the opposite side of the conductive gel layer 12) of the sheet material 2. Furthermore, the connection position may be inside the sheet material 2. In this way, it is possible to manufacture the bioelectrode from the electrode 20.

In the electrode 20, the conductive gel layer 12 is disposed directly on the conductive portion 4. Thus, the above-described conductive layer is not necessary. The present embodiment can provide a bioelectrode having a more simplified structure than that of Embodiments 1 and 2 including the above-described conductive layer. Therefore, the manufacturing process can be further simplified.

### Embodiment 4

Further embodiments of the present disclosure will be described below. The present embodiment is the same as Embodiment 3 except that the sheet material is impregnated with plural types of conductive coating materials. The matters overlapping those of the above-described embodiments may not be described again.

FIG. 8 is a cross-sectional view schematically showing the structure of the electrode according to Embodiment 4. An electrode 30 has the sheet material 2 and the conductive gel layer 12 disposed on the surface of the sheet material 12. The sheet material 2 has the conductive portion 4 made of two kinds of conductive coating materials.

That is, in the thickness direction of the sheet material 2, a portion close to the first surface thereof is impregnated with a first conductive coating material 15. A portion close to the second surface is impregnated with a second conductive coating material 25. A portion (first impregnated portion) of the sheet material 2 impregnated with the first conductive coating material 15 and a portion (second impregnated portion) impregnated with the second conductive coating material 25 may be overlapped in the thickness direction of the sheet material 2, for example, by the first conductive coating material 15 being impregnated up to the second impregnated portion. In the conductive portion 4, the first conductive coating material 15 and the second conductive coating material 25 are sufficiently impregnated in the thickness direction of the sheet material 2 as described above.

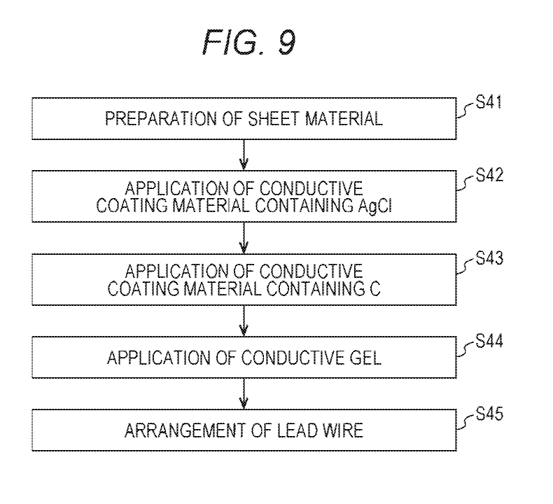

In the present embodiment, the bioelectrode can be manufactured by the following method. FIG. 9 is a flowchart of a method of manufacturing the bioelectrode using the electrode 30.

First, the sheet material 2 is prepared (S41). It is possible to prepare the sheet material 2 in the same manner as in Step S21 described above.

Next, the first conductive coating material 15 is applied to the first surface of the sheet material 2 (S42). The first conductive coating material 15 is, for example, the above-mentioned conductive coating material containing silver chloride (AgCl-containing conductive coating material).

Next, the second conductive coating material 25 is applied to the second surface of the sheet material 2 (S43). The second conductive coating material 25 is, for example, the above-mentioned ink containing carbon particles (C-containing conductive coating material).

The coating method of the first and second conductive coating materials 15 and 25 can be appropriately determined from known coating methods within a range in which the conductive coating material can be impregnated into the sheet material 2 from the surface of the sheet material 2 to a sufficient depth. The first impregnated portion and the second impregnated portion impregnated with the first and second conductive coating materials 15 and 25 in the sheet material 2 only have to be at least in contact with each other in the thickness direction of the sheet material 2. However, it is preferable that the first impregnated portion and the second impregnated portion overlap each other from the viewpoint of realizing the electrically conductive portion 4 having sufficient conductivity.

Then, the conductive gel is applied to the surface of the sheet material 2 impregnated with the first and second conductive coating materials 15 and 25. In this manner, the conductive gel layer 12 is produced (S44). The conductive gel can be applied in the same manner as in Step S25 described above.

Next, the lead wire is placed on the sheet material 2 (S45). The connection position of the lead wire in the sheet material 2 can be appropriately determined within a range of sufficiently conducting to the conductive portion, and for example, it can be in the same manner as in Step S34 in the above-described Embodiment 3. In this way, it is possible to manufacture the bioelectrode from the electrode 30.

In the electrode 30, the conductive gel layer 12 is disposed directly on the conductive portion 4. Thus, the above-described conductive layer is not necessary. The present embodiment can also provide the bioelectrode having a more simplified structure than that of Embodiments 1 and 2 including the above-described conductive layer as in Embodiment 3 described above. Therefore, the manufacturing process can be further simplified.

### [Modification of Embodiments 3 and 4]

The electrodes 20 and 30 may further include a conductive layer disposed at a position overlapping the conductive portion on the surface of the sheet material 2. According to such a structure, it is possible to form the electrode having the same structure as that of Embodiment 1. Therefore, it is possible to form the same bioelectrode as in Embodiment 2.

The bioelectrode in Embodiments 3 and 4 further includes the conductive layer disposed on the conductive portion 4 between the conductive portion 4 and the conductive gel layer 12, and the silver chloride layer disposed on the conductive layer. The lead wire may be electrically connected to the conductive portion 4 or the conductive layer. According to such a structure, it is possible to form the electrode having the same structure as that of Embodiment 2. As described above, in the present disclosure, the lead wire may be electrically connected directly to the conductive portion 4. Alternatively, the lead wire may be electrically connected to the conductive portion through another conductive element like the conductive layer.

The method of manufacturing the electrodes 20 and 30 may further include a step of forming the conductive layer at a position overlapping the conductive portion 4 on the surface of the sheet material 2. According to such a structure, it is possible to manufacture the electrode having the same structure as that of Embodiment 1.

The method of manufacturing the bioelectrode in Embodiments 3 and 4 further includes a step of forming the conductive layer on the conductive portion 4 and a step of forming the silver chloride layer on the conductive layer. The step of forming the conductive gel layer 12 is a step of forming the conductive gel layer 12 on the silver chloride layer. The step of electrically connecting the lead wire may be a step of electrically connecting the lead wire to the conductive layer or the conductive part. According to such a structure, it is possible to manufacture the bioelectrode having the same structure as that of Embodiment 2.

### [Summary of Embodiments 3 and 4]

The electrode according to an aspect 12 of the present disclosure includes the sheet material. The sheet material includes the conductive portion which is continuous so that the sheet material has electrical conductivity in the thickness direction thereof. The conductive portion includes the electrical conductor distributed in the conductive portion so that the sheet material has electrical conductivity in the thickness direction.

According to the above structure, as with the electrode according to Embodiment 1, it is possible to conduct from both front and back surfaces of the sheet material. In addition, it is possible to form the electrode more simply than the electrode according to Embodiment 1.

In the electrode according to an aspect 13 of the present disclosure, the electrical conductor of the aspect 12 may contain carbon particles.

The above structure is more effective from the viewpoint of improving the corrosion resistance of the electrode and the viewpoint of suitably adjusting the electrical conductivity of the conductive portion.

In the electrode according to an aspect 14 of the present disclosure, the electrical conductor of Embodiment 12 or 13 may contain silver chloride.

The above structure is more effective from the viewpoint of applying the electrode to the bioelectrode.

The bioelectrode according to an aspect 15 of the present disclosure includes the electrode according to any one of the aspects 12 to 14, the conductive gel layer disposed on the conductive portion, and the lead wire electrically connected to the conductive portion.

According to the above structure, as with the bioelectrode according to Embodiment 2, it is possible to increase the degree of freedom in the structure of the bioelectrode. In addition, it is possible to form the bioelectrode more simply than that of the bioelectrode according to Embodiment 2.

A bioelectrode according to an aspect 16 of the present disclosure may be the electrode for measuring the electrocardiogram.

The method of manufacturing the electrode according to an aspect 17 of the present disclosure includes the step of permeating a sheet material having liquid permeability with a conductive coating material containing an electrical conductor, to form the conductive portion which is continuous so that the sheet material has electrical conductivity in a thickness direction thereof.

According to the above structure, as with the method of manufacturing the electrode according to Embodiment 1, it is possible to manufacture the electrode which can easily conduct from any surface of the sheet material. In addition, it is possible to manufacture such an electrode more simply than the method of manufacturing the electrode according to Embodiment 1.

In the method of manufacturing method of the electrode according to an aspect 18 of the present disclosure, the carbon particles may be used as the electrical conductor of the aspect 17.

The above structure has the same effect as the aspect 13 described previously.

In the method for manufacturing the electrode according to an aspect 19 of the present disclosure, the silver chloride may be used as the electrical conductor of Embodiment 17 or 18.

The above structure has the same effect as the aspect 14 described previously.

The method of manufacturing the bioelectrode according to an aspect 20 of the present disclosure includes the steps of forming the conductive gel layer on the conductive portion of the electrode manufactured by the method of manufacturing the electrode according to any one of Embodiments 17 to 19, and electrically connecting the lead wire to the conductive portion.

According to the above structure, as with the method of manufacturing the bioelectrode according to Embodiment 2, it is possible to increase the degree of freedom in the structure of the bioelectrode. In addition, it is possible to manufacture the bioelectrode more simply than the method of manufacturing the bioelectrode according to Embodiment 2.

According to Embodiments 3 and 4, it is possible to further increase the degree of freedom in the structure of the electrode and the bioelectrode in addition to the effects of Embodiments 1 and 2.

### EXAMPLES

### Example 1

As the sheet material, “MALIX#20451FLV” which is the nonwoven fabric manufactured by Unitika Ltd. was used. The silver metal layer of 1000 Å (0.1 μm) thickness was formed on the first surface of the nonwoven fabric by vacuum evaporation. Carbon ink (also referred to as “CI”) was applied to the second surface of the sheet material by a screen coating method. “UCC-2” manufactured by Nippon Graphite Industries Co., Ltd. was used as the CI. A layer of CI was formed on the second surface of the sheet material by permeating the sheet material with the CI. A thickness of the CI layer was 3 μm. An electrode A1 was manufactured in this manner.

Electrodes A2 to A4 were manufactured by the same method as for the electrode A1 except that each thickness of the CI layer was changed to 5 μm, 10 μm, and 15 μm by changing coating conditions of the CI.

### [Evaluation of Electrode]

For each of the electrodes A1 to A4, electrical resistance values of the metal layer, the conductive portion, and the whole electrode were measured. The metal layer is the silver-deposited film on the surface of the sheet material. The electrical resistance value of the metal layer is the electrical resistance value between arbitrary two points (between 10 mm) on the surface of the metal layer, and was measured with a digital multimeter FLUKE85 manufactured by Fluke Corporation.

The conductive portion is a portion where the CI of the sheet material is applied and permeated, and is the conductive portion and the conductive coating film in the previously described embodiment. The electrical resistance value of the conductive portion is the electrical resistance value between arbitrary two points (between 10 mm) on the surface of the conductive coating film, and was measured with the digital multimeter FLUKE85 manufactured by Fluke Corporation.

The electrical resistance value of the whole electrode is the electrical resistance value between the conductive coating film and the metal layer in the thickness direction of the electrode. When measuring the electrical resistance value, first, the electrode of about 100 mm×100 mm is placed on a gold-plated metal plate, and a columnar gold-plated metal (f30 mm×40 mmh, 225 g) is placed thereon. Thus, the electrode is sandwiched between the gold-plated metal plate and the columnar gold-plated metal. Then, the electrical resistance between the metal plate and the gold-plated metal was measured with the digital multimeter FLUKE85 manufactured by Fluke Corporation. Results are shown in Table 1.

TABLE 1

| | | Elec- | Elec- | Elec- | Elec- |
|---|---|---|---|---|---|
| | | trode | trode | trode | trode |
| | | A1 | A2 | A3 | A4 |
| Thickness of metal layer (Å) | | 1000 | 1000 | 1000 | 1000 |
| Thickness of CI layer (μm) | | 3 | 5 | 10 | 15 |
| Electrical | Metal layer | 0.6 | 0.7 | 0.6 | 0.6 |
| resistance | (Ω/10 mm) | | | | |
| value | Conductive | 758 | 329 | 251 | 272 |
| | portion | | | | |
| | (Ω/10 mm) | | | | |
| | Whole (Ω) | ∞ | 120-360 | 49.1 | 37.8 |

Conduction was not detected in the electrode A1. This is thought to be because the CI layer was thin and permeation of the CI into the sheet material was insufficient, and thus a sufficiently energizable conductive portion was not formed.

The conduction was detected in the electrode A2. In the electrodes A3 and A4, sufficient and stable conduction was detected in the whole electrode.

### Example 2

Silver chloride paste was applied to the surface of the metal layer of the electrode A3 by the screen method, and the coating film was dried to produce the silver chloride layer having a thickness of 10 to 15 The silver chloride paste is a composition containing “Silver Chloride (I) special grade” manufactured by Kishida Chemical Co., Ltd. and “Nichigo-POLYESTER LP-035” manufactured by Nippon Synthetic Chemical Industry Co., Ltd. as an ink binder (“Nichigo-POLYESTER” is a registered trademark of the company). In the screen method, a 180-mesh polyester screen mask was used. “Technogel” manufactured by Sekisui Plastics Co., Ltd. (“Technogel” is a registered trademark of the company) was attached to the surface of the silver chloride layer. The Technogel is the conductive gel layer in the previously described embodiment. In this manner, a bioelectrode B1 was manufactured.

In addition, the bioelectrode B2 was manufactured by the same method as for the bioelectrode B1 except that the electrode A4 was used instead of the electrode A3.

### Example 3

The nonwoven fabric was impregnated with the conductive ink by the dipping method to prepare an electrode A5. ELTAS EC5045C manufactured by Asahi Kasei Corporation was used as the nonwoven fabric. A mixed ink containing carbon, metallic silver, and silver chloride having the following composition was used as the conductive ink. The electrode A5 has the conductive portion containing carbon, metallic silver, and silver chloride as the electrical conductor. Then, the Technogel was attached as the conductive gel layer to the surface of the conductive portion. Thus, the bioelectrode B3 was manufactured.

### (Composition of Conductive Ink)

| | | |
|---|---|---|
| UCC-2 | 79 | mass % |
| Silver chloride (I) special grade | 5 | mass % |
| Silver powder | 16 | mass % |

“Ag-4-8F” (manufactured by DOWA Electronics Materials Co., Ltd. shape: spherical, average particle size: 2.2 μm) was used as “silver powder”.

### Example 4

The electrode A6 was manufactured by the same method as in Example 3 except that the CI was applied to the first surface of the nonwoven fabric and the conductive ink was applied to the second surface each in an amount of 5 to 8 g/m² by the gravure coating method. The CI layer and the conductive ink layer in the electrode A6 were sufficiently in contact with each other in the thickness direction of the nonwoven fabric. Parts of both layers overlapped each other. In this way, the conductive portion including the CI layer and the conductive ink layer was formed. Then, the Technogel was attached as the conductive gel layer to the surface of the conductive portion. Thus, the bioelectrode B4 was manufactured.

### [Evaluation of Bioelectrode]

A test of electrical properties according to the electrocardiography method was performed using each of the bioelectrodes B1 and B2. More specifically, impedance (AC impedance (10 Hz)) and offset voltage (DC offset voltage) of the bioelectrode were measured. The impedance and the offset voltage of the bioelectrode are measured by the American National Standards Institute (ANSI)/the Association for the Advancement of Medical Instrumentation (AAMI) standard EC12.

More specifically, the surfaces of the gels attached to two electrodes of each of the bioelectrodes B1 to B4 were bonded to each other. In this manner, a measurement probe terminal was connected to each of the bioelectrodes. That is, each of the bioelectrodes B1 to B4 was cut into two strips of 2.5 cm×4.5 cm. An evaluation test piece was prepared by bonding surfaces of the cut conductive gels to each other. The measurement probe terminal was connected to the evaluation test piece.

Then, the impedance and the offset voltage were measured using the evaluation test piece. A Surface Electrode Analysis Meter ECG tester manufactured by CALM Co., Ltd. was used for measuring the impedance and the offset voltage. The tests of the electrical properties were respectively carried out with test number N=3. An average value of measured values was defined as an evaluation value.

The results are shown in Table 2. In the above standard, the AC impedance (10 Hz) may be 2 kΩ or less on average. Further, the DC offset voltage may be 100 mV or less on average.

TABLE 2

| Bioelectrode No. | Impedance (Ω) | Offset voltage (mV) |
|---|---|---|
| B1 | 151 | 5.5 |
| B2 | 275 | 22.9 |
| B3 | 413 | 1.3 |
| B4 | 550 | 1.1 |

As is apparent from Table 2, the impedance and the offset voltage of the bioelectrodes B1 to B4 both satisfied the standard of the American National Standards Institute (ANSI)/the Association for the Advancement of Medical Instrumentation (AAMI). From the results, it is understood that all the bioelectrodes B1 to B4 can be applied to a base material for electrocardiogram measurement.

### [Discussion]

From the above examples, it is considered that the electrode and the bioelectrode of the present embodiments have the following advantages.

For the electrode for the general bioelectrode, it is necessary to apply an additional method such as a through-hole method so that the sheet material has electrical conductivity between a plurality of surfaces thereof. In the through-hole method, it is necessary to form a conduction path by filling the through-hole formed in the electrode with the conductor. In contrast, the electrode and the bioelectrode of the above embodiments are manufactured by printing a conductive material so as to obtain electrical conductivity between the plurality of such surfaces. Therefore, the electrical conductivity between the plurality of surfaces can be obtained without applying the additional method such as the through-hole method.

One surface of the electrode can be utilized as a current collecting position for providing an electrical contact. Thus, in an assembly such as a bioelectrode, it is possible to have more options for its use form and usage.

In the bioelectrode, it is possible to cover the entire metal layer as the conductive layer. Therefore, exposure of the metal layer can be prevented. In the conductive portion, the conductor such as carbon which is stable against corrosion can be used. As a result, it is possible to prevent corrosion of the conductor and conduction failure due to the corrosion.

In the electrode and the bioelectrode, it is also possible to make the conductive portion play the role of the conductive layer instead of disposing the conductive layer. According to such a configuration, it is possible to form the electrode and the bioelectrode having a simpler structure in addition to the above advantages.

The electrodes of the present disclosure may be the following first to eighth electrodes.

The first electrode is the electrode including the sheet material and the conductive portion having electrical conductivity in the thickness direction of the sheet material, and the conductive portion includes the conductor which is distributed so that the conductive portion has electrical conductivity in the thickness direction of the sheet material.

The second electrode is the first electrode, wherein the electrical conductor contains the carbon particles.

The third electrode is the first or second electrode, wherein the electrical conductor includes silver chloride.

The fourth electrode is any one of the first to third electrodes, further including the conductive layer disposed at the position overlapping the conductive portion on the surface of the sheet material.

The fifth electrode is the fourth electrode, wherein the conductive layer includes a metal layer.

The sixth electrode is the fifth electrode, wherein the metal layer is the metal-deposited layer.

The seventh electrode is the fifth or sixth electrode, wherein the metal material of the metal layer is one or more metal materials selected from the group consisting of gold, silver, copper, nickel, tin, aluminum, zinc, indium tin oxide, and titanium.

The eighth electrode is any one of the first to seventh electrodes, wherein the sheet material is one or more members selected from the group consisting of the nonwoven fabric, the woven fabric, the knitted material, the paper, the synthetic resin net, and the metal net.

The bioelectrodes of the present disclosure may be the following first to third bioelectrodes.

The first bioelectrode includes any one of the first to eighth electrodes, the conductive gel layer disposed on the conductive portion, and the lead wire electrically connected to the conductive portion.

The second bioelectrode is the first bioelectrode, further including the conductive layer disposed on the conductive portion between the conductive portion and the conductive gel layer, and the silver chloride layer disposed on the conductive layer, wherein the lead wire is electrically connected to the conductive portion or the conductive layer.

The third bioelectrode is the first or second bioelectrode, which is the electrode for measuring the electrocardiogram.

The methods of manufacturing the electrode of the present disclosure may be the following first to fourth methods of manufacturing the electrode.

The first method of manufacturing the electrode is the method of manufacturing the electrode, including the step of permeating the sheet material having liquid permeability with the conductive coating material containing the electrical conductor, to form the conductive portion having electrical conductivity in the thickness direction of the sheet material.

The second method of manufacturing the electrode is the first method of manufacturing the electrode, wherein the carbon particles are used as the electrical conductor.

The third method of manufacturing the electrode is the first or second method of manufacturing the electrode, wherein the silver chloride is used as the electrical conductor.

The fourth method of manufacturing the electrode is any one of the first to third methods of manufacturing the electrode, further including the step of forming the conductive layer at the position overlapping the conductive portion on the surface of the sheet material.

The method of manufacturing the bioelectrode of the present disclosure may be the following first or second method of manufacturing the bioelectrode.

The first method of manufacturing the bioelectrode includes the step of forming the conductive gel layer on the conductive portion of the electrode manufactured by any one of the first to fourth methods of manufacturing the electrode, and the step of electrically connecting the lead wire to the conductive portion.

The second method of manufacturing the bioelectrode is the first method of manufacturing the bioelectrode, further including the step of forming the conductive layer on the conductive portion and the step of forming the silver chloride layer on the conductive layer, wherein the step of forming the conductive gel layer is the step of forming the conductive gel layer on the silver chloride layer, and the step of electrically connecting the lead wire is the step of electrically connecting the lead wire to the conductive layer or the conductive portion.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. An electrode comprising:
a sheet material, wherein
the sheet material comprises a conductive portion which is continuous so that the sheet material has electrical conductivity in a thickness direction thereof, wherein
the conductive portion comprises an electrical conductor distributed in the conductive portion so that the sheet material has electrical conductivity in the thickness direction thereof.

2. The electrode according to claim 1, wherein the electrical conductor comprises carbon particles.

3. The electrode according to claim 1, wherein the electrical conductor comprises silver chloride.

4. The electrode according to claim 1, further comprising a conductive layer disposed at a position overlapping the conductive portion on a surface of the sheet material.

5. The electrode according to claim 4, wherein the conductive layer comprises a metal layer.

6. The electrode according to claim 5, wherein the metal layer is a metal deposited layer.

7. The electrode according to claim 5, wherein a metal material of the metal layer is one or more metal materials selected from a group consisting of gold, silver, copper, nickel, tin, aluminum, zinc, indium tin oxide, and titanium.

8. The electrode according to claim 1, wherein the sheet material is one or more members selected from a group consisting of a nonwoven fabric, a woven fabric, a knitted material, a paper, a synthetic resin net, and a metal net.

9. A bioelectrode comprising the electrode according to claim 1, a conductive gel layer disposed on the conductive portion, and a lead wire electrically connected to the conductive portion.

10. The bioelectrode according to claim 9, further comprising a conductive layer disposed on the conductive portion between the conductive portion and the conductive gel layer, and a silver chloride layer disposed on the conductive layer, wherein
the lead wire is electrically connected to the conductive portion or the conductive layer.

11. The bioelectrode according to claim 9, which is an electrode for measuring an electrocardiogram.

12. A method of manufacturing an electrode, comprising a step of permeating a sheet material having liquid permeability with a conductive coating material containing an electrical conductor, to form a conductive portion which is continuous so that the sheet material has electrical conductivity in a thickness direction thereof.

13. The method of manufacturing the electrode according to claim 12, wherein carbon particles are used as the electrical conductor.

14. The method of manufacturing the electrode according to claim 12, wherein silver chloride is used as the electrical conductor.

15. The method of manufacturing the electrode according to claim 12, further comprising a step of forming a conductive layer at a position overlapping the conductive portion on a surface of the sheet material.

16. A method of manufacturing a bioelectrode, comprising:
a step of forming a conductive gel layer on the conductive portion of the electrode manufactured by the method of manufacturing the electrode according to claim 12; and
a step of electrically connecting a lead wire to the conductive portion.

17. The method of manufacturing the bioelectrode according to claim 16, further comprising:
a step of forming a conductive layer on the conductive portion; and
a step of forming a silver chloride layer on the conductive layer, wherein
the step of forming the conductive gel layer is a step of forming a conductive gel layer on the silver chloride layer, and
the step of electrically connecting the lead wire is a step of electrically connecting the lead wire to the conductive layer or the conductive portion.
